(19) United States
(12) Patent Application Publication
(10) Pub. No.: US 2026/0325812 A1
(43) Pub. Date: Oct. 22, 2026

(54) ASYMMETRICALLY OPERATING TWO WAY PRESSURE ACTIVATED VALVE AND METHODS OF USING SAME

(71) Applicant: NP Medical Inc., Clinton, MA (US)

(72) Inventors: John Damarati, Marlborough, MA (US); Todd Chelak, Pelham, NH (US); Nicholas Illsley, Sterling, MA (US)

(21) Appl. No.: 19/486,202

(22) PCT Filed: May 26, 2023
(86) PCT No.: PCT/US2023/023665

§ 371 Date: Nov. 20, 2025

(51) Int. Cl. A61M 39/02

(52) U.S. Cl. CPC A61M 39/0247; A61M 2039/0258; A61M 2039/027

(57) ABSTRACT

A medical valve includes a body having an internal fluid path extending from a first opening to a second opening. A valve member located within the fluid path has a closed mode, and a first and second open mode. When in the first open mode, an outer section of the valve member deforms toward a distal end, and when in the second open mode, a portion of the outer section deforms toward a proximal end. The outer section of the valve member seals against a seal surface when in the closed mode. The outer section of the valve member deforms in an asymmetrical manner such that the first portion of the outer section moves toward the proximal end of the body an amount greater than the second portion as the valve member transitions toward the second open mode.

### TECHNICAL FIELD

The present invention relates to valving systems, and more particularly to diaphragm based valving devices and systems with improved flow.

### BACKGROUND ART

In instances in which a patient will need regular administration of fluid or medications (or regular withdrawal of fluids/blood), catheters are often inserted into the patient and used to administer the fluids/medications. The catheter may remain in the patient for extended periods of time (several hours to several days or longer). Additionally, an extension tube, an administration set, or both may be connected to the catheter to facilitate use of the catheter and connection of a medical implement (e.g., a syringe).

The extension tube, administration set, medical implement, or similar vascular access device may include a medical valving device. In general terms, medical valving devices often act as a means for selectively preventing fluid flow within a fluid system connected to a patient's vasculature. Consequently, a medical valve permits the patient's vasculature to be freely accessed while also controlling the conditions under which fluid flow is to occur. The medical valve may be an element of a luer activated valve (with or without a swabable septum) and/or a pressure activated valve (similarly with or without a swabable septum). An issue with many prior art medical valves (particularly two way pressure activated valves that utilize a diaphragm with a slit-style aperture) is that fluid flow through the aperture may be constricted.

### SUMMARY OF THE INVENTION

In accordance with one embodiment of the invention, a medical valve for use within a device in fluid communication with a patient's vasculature may have a body that defines the structure of the medical valve. The body has a first opening at a proximal end of the body, a second opening at a distal end of the body, and an internal fluid path extending from the first opening to the second opening. The valve may also have a valve member located within the internal fluid path. The valve member may have an outer section and an inner section. When in a closed mode, the valve member may prevent fluid flow through the medical valve. When in a first open mode the valve member may allow fluid flow from the first opening to the second opening. When in a second open mode the valve member may allow fluid flow from the second opening to the first opening. The outer section may deform toward the distal end as the valve member transitions from the closed mode toward the first open mode in the presence of a distally directed force or pressure, and at least a first portion of the outer section may deform toward the proximal end as the valve member transitions from the closed mode toward the second open mode in the presence of a proximally directed force. The valve member transitions from the second open mode to either the closed mode or the first open mode in the presence of a distally directed force or pressure.

On the body, the valve may have a seal surface and the outer section of the valve member may seal against the seal surface when in the closed mode. Within the fluid path, the valve may have a positioning structure and a support structure. The positioning structure may be located distal to the valve member and maintain the position of the inner section of the valve member in the presence of a continually applied distally directed force or pressure. The support structure may be located proximal to the valve member and support at least a second portion of the valve member in the presence of a continually applied proximally directed force or pressure. The outer section of the valve member may deform in an asymmetrical manner such that at least the first portion of the outer section of the valve member moves toward the proximal end of the body an amount greater than the second portion as the valve member transitions toward the second open mode.

In accordance with some embodiments, the body may include an inlet housing and an outlet housing. The first opening may be located in the inlet housing, and the second opening may be located in the outlet housing. The seal surface and the support structure may be located on the inlet housing. The positioning structure may be located in the outlet housing and may position the valve member between the inlet and outlet housing. The outer section of the valve member may deform toward the outlet housing as the valve member transitions toward the first open mode, and at least a portion of the outer section of the valve member may deform toward the inlet housing as the valve transitions toward the second open mode.

In additional embodiments, the outlet housing may include at least one protrusion. The protrusion(s) may position the valve member when in the first open mode and may, at least partially, define one or more flow channels through which fluid may flow. The inlet housing may have at least one support arm that may support the valve member when in the second open mode and may, at least partly, define one or more flow channels through which fluid may flow. The at least a first portion of the outer section may not be supported by the support structure. The second portion of the valve member may include a second portion of the outer section and at least a portion of the inner section of the valve member. The valve member may be a flexible diaphragm and/or a pressure activated valve.

In some embodiments, the proximally directed force required to transition the valve member from the closed mode to the second open mode may be greater than the distally directed pressure required to transition the valve member to the first open mode. Additionally or alternatively, the proximally directed force required to transition the valve member from the closed mode to the second open mode may be greater than a patient's venous pressure. The first opening may connect to a tube of an extension set or an IV set. The amount of force required to move the outer section of the valve member past the edge of the sealing surface as the valve member transitions from the second open mode to either the closed mode or the first open mode may be based on a number of factors including, but not limited to, the amount of radial interference between the outer section and the sealing surface, the surface geometry and/or roughness of the inner wall of the medical valve, the thickness and/or durometer of the valve member, the angles between the inner wall and the edge of valve member, and the lubricity of the surface of the valve member or inner wall.

In accordance with further embodiments, a fluid management device in communication with a patient's vasculature may include a medical valve and tube having a first and a second end. The medical valve may have a body that defines the structure of the medical valve, and has a first opening at a proximal end of the body, a second opening at a distal end of the body, and an internal fluid path extending from the first opening to the second opening. The valve may also have a valve member located within the internal fluid path. The valve member may have an outer section and an inner section. When in a closed mode, the valve member may prevent fluid flow through the medical valve. When in a first open mode the valve member may allow fluid flow from the first opening to the second opening. When in a second open mode the valve member may allow fluid flow from the second opening to the first opening. The outer section may deform toward the distal end as the valve member transitions from the closed mode toward the first open mode in the presence of a distally directed force or pressure, and at least a first portion of the outer section may deform toward the proximal end as the valve member transitions from the closed mode toward the second open mode in the presence of a proximally directed force or pressure. The valve member transitions from the second open mode to either the closed mode or the first open mode in the presence of a distally directed force or pressure.

On the body, the valve may have a seal surface and the outer section of the valve member may seal against the seal surface when in the closed mode. Within the fluid path, the valve may have a positioning structure and a support structure. The positioning structure may be located distal to the valve member and maintain the position of the inner section of the valve member in the presence of a continually applied distally directed force or pressure. The support structure may be located proximal to the valve member and support at least a second portion of the valve member in the presence of a continually applied proximally directed force or pressure. The outer section of the valve member may deform in an asymmetrical manner such that at least the first portion of the outer section of the valve member moves toward the proximal end of the body an amount greater than the second portion as the valve member transitions toward the second open mode. The first end of the tube may fluidly connect to the first opening of the medical valve, and the second end of the tube may be connected to a female luer connector. The female luer connector may connect to a medical implement that introduces fluid into or draws fluid from the patient through the medical valve. The distal end of the medical valve may connect to a catheter.

In accordance with additional embodiments, a method for controlling fluid flow through a medical valve may include providing a medical valve. The medical valve may have a body that defines the structure of the medical valve, and has a first opening at a proximal end of the body, a second opening at a distal end of the body, and an internal fluid path extending from the first opening to the second opening. The valve may also have a valve member located within the internal fluid path. The valve member may have an outer section and an inner section. When in a closed mode, the valve member may prevent fluid flow through the medical valve. When in a first open mode the valve member may allow fluid flow from the first opening to the second opening. When in a second open mode the valve member may allow fluid flow from the second opening to the first opening. The outer section may deform toward the distal end as the valve member transitions from the closed mode toward the first open mode in the presence of a distally directed force or pressure, and at least a first portion of the outer section may deform toward the proximal end as the valve member transitions from the closed mode toward the second open mode in the presence of a proximally directed force or pressure. The valve member transitions from the second open mode to either the closed mode or the first open mode in the presence of a distally directed force or pressure.

On the body, the valve may have a seal surface and the outer section of the valve member may seal against the seal surface when in the closed mode. Within the fluid path, the valve may have a positioning structure and a support structure. The positioning structure is located distal to the valve member and maintains the position of the inner section of the valve member in the presence of a continually applied distally directed force or pressure. The support structure is located proximal to the valve member and supports at least a second portion of the valve member in the presence of a continually applied proximally directed force or pressure. The outer section of the valve member may deform in an asymmetrical manner such that at least the first portion of the outer section of the valve member moves toward the proximal end of the body an amount greater than the second portion as the valve member transitions toward the second open mode.

The method may then generate a pressure or force on the valve member to transition the valve member toward the first open mode or the second open mode, and transfer fluid through the internal fluid path and around at least a portion of the valve member. In some embodiments, the pressure/force is a distally directed pressure and the valve member transitions toward the first open mode to allow fluid transfer to the patient and/or transition the valve member to the closed mode. In other embodiments, the pressure/force is a proximally directed force and the valve member transitions toward the second open mode to allow fluid transfer from the patient.

### BRIEF DESCRIPTION OF THE DRAWINGS

The foregoing features of embodiments will be more readily understood by reference to the following detailed description, taken with reference to the accompanying drawings, in which:

FIG. 1 schematically shows a perspective view of a pressure activated valve in accordance with various embodiments of the present invention;

FIG. 2A schematically shows a cross-section view of the valve shown in FIG. 1 in the closed mode, in accordance with some embodiments of the present invention;

FIG. 2B schematically shows a cross-section view of the valve shown in FIG. 1 in the first open mode, in accordance with some embodiments of the present invention

FIG. 2C schematically shows a cross-section view of the valve shown in FIG. 1 in the second open mode, in accordance with some embodiments of the present invention;

FIGS. 3A-3C schematically show various perspective exploded views of the pressure activated valve shown in FIG. 1, in accordance with some embodiments of the present invention;

FIG. 4 schematically shows an exploded cross-sectional view of the pressure activated valve shown in FIG. 1, in accordance with various embodiments of the present invention;

FIGS. 5A-5C schematically show a bottom view of a top portion/inlet housing of the valve shown in FIG. 1, in accordance with various embodiments of the present invention;

FIG. 6 schematically shows an extension set with a pressure activated valve, in accordance with embodiments of the present invention.

### DETAILED DESCRIPTION OF SPECIFIC EMBODIMENTS

In illustrative embodiments, a medical valve for use within devices in fluid communication with a patient's vasculature has an internal valve member located within an internal fluid path of the medical valve. The housing of the valve has a support structure that supports a portion of the valve member such that the valve member deforms asymmetrically in the presence of a proximally directed pressure or force within the valve. This, in turn, allows sufficient retrograde flow through the valve or more specifically, around the valve member. Details of illustrative embodiments are discussed below.

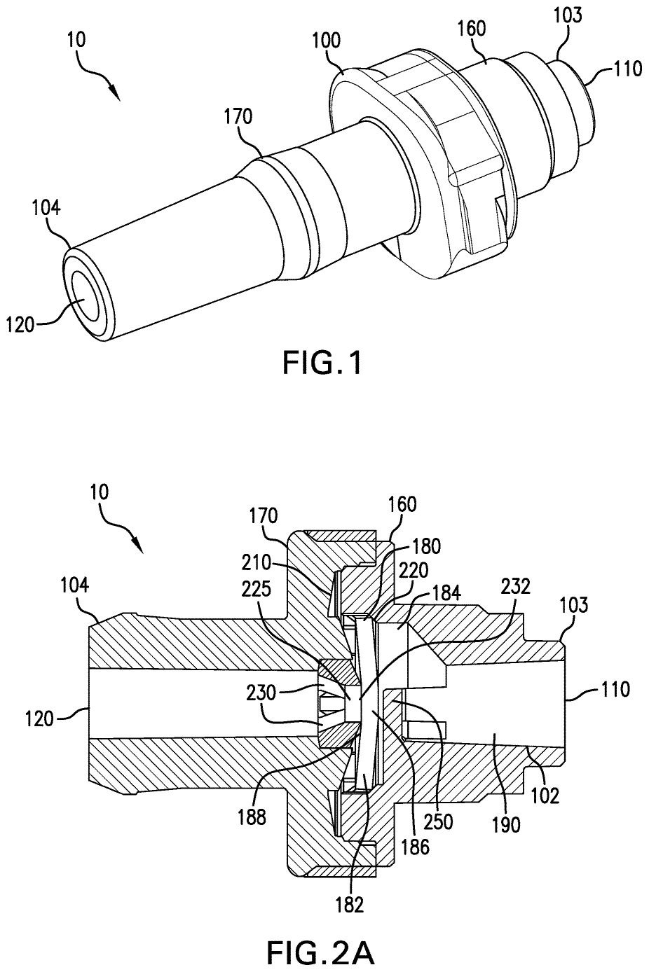

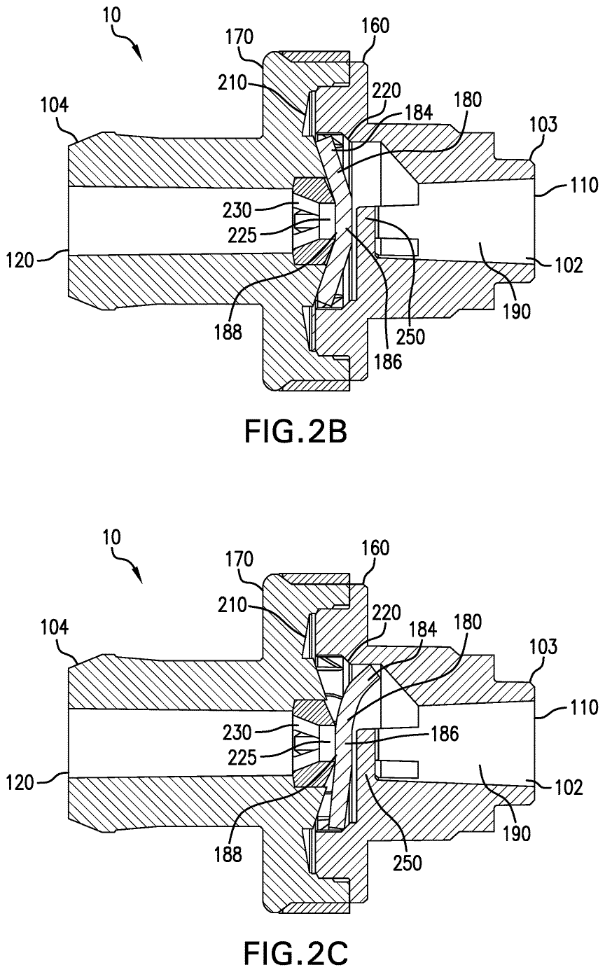

FIG. 1 schematically shows a perspective view of a medical valve 10 in accordance with some embodiments of the present invention. The valve 10 has a housing 100 forming an interior having a proximal port 110 (e.g., an inlet or first opening) for receiving a medical instrument (not shown), and a distal port 120 (e.g., an outlet or section opening). The valve 10 has a first open mode that permits forward fluid flow through the valve 10 (e.g., from the proximal port 110 to the distal port 120), a second open mode that permits retrograde flow through the valve 10 (e.g., from the distal port 120 toward the proximal port 110), and a closed mode that prevents fluid flow through the valve 10. To that end, the interior contains a valve mechanism/valve member 180 (FIGS. 2A-2C) that selectively controls (i.e., allows/permits) fluid flow through the valve 10. The fluid passes through a complete fluid path 190 (FIGS. 2A-2C) that extends between the proximal port 110 and the distal port 120.

It should be noted that although much of the discussion herein refers to the proximal port 110 as an inlet, and the distal port 120 as an outlet, as discussed in greater detail below and in some embodiments, the proximal and distal ports 110 and 120 also may be respectively used as inlet and outlet ports. Discussion of these ports in either configuration therefore is for illustrative purposes only.

FIG. 2A schematically shows a cross section of the valve shown in FIG. 1 when in the closed mode, FIG. 2B schematically shows a cross section of the valve 10 in the first open mode, and FIG. 2C schematically shows a cross section of the valve 10 in the second open mode. As shown, the housing 100 includes an inlet housing 160 and an outlet housing 170, which connect together to form the interior of the medical valve 10. Within the interior, the medical valve 10 has a valve mechanism 180 located within the fluid path 190 through the housing 100. The inlet housing 160 and the outlet housing 170 may be joined together in a variety of ways, including a snap-fit connection, ultrasonic welding, plastic welding, or other methods conventionally used in the art.

Within the interior, the body/housing 100 may have an inner wall 102 that extends along at least a portion of the longitudinal axis of the valve 10. The inner wall 102 forms/defines the internal fluid path 190 that extends through the valve 10 from the inlet 110 to the outlet 120. As discussed in greater detail below, the body/housing 100 (e.g., the outlet body/housing 170) may have a base portion 210 that extends inward from the inner wall 102. It should be noted that, although the fluid path 190 is shown as having a circular cross-sectional shape, other embodiments may have fluid paths with different cross-sectional shapes.

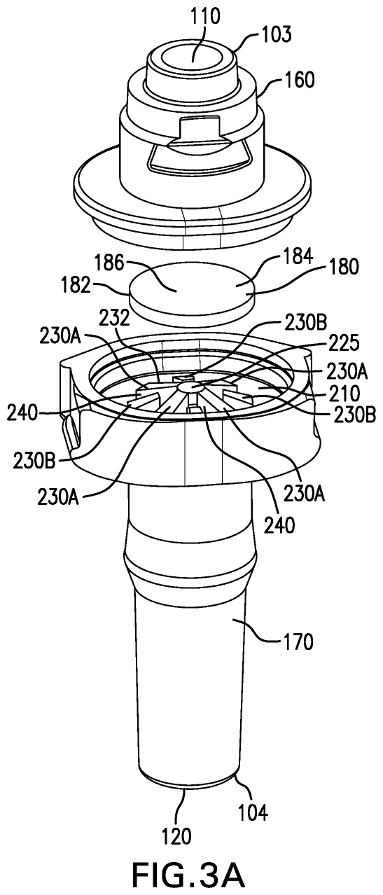

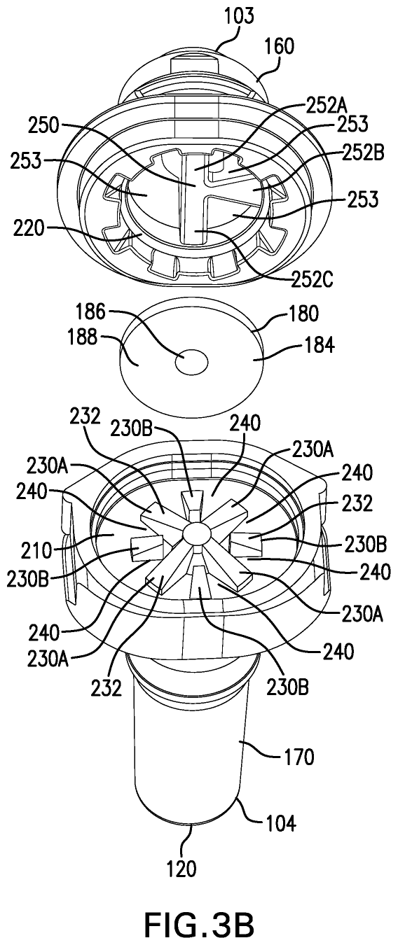

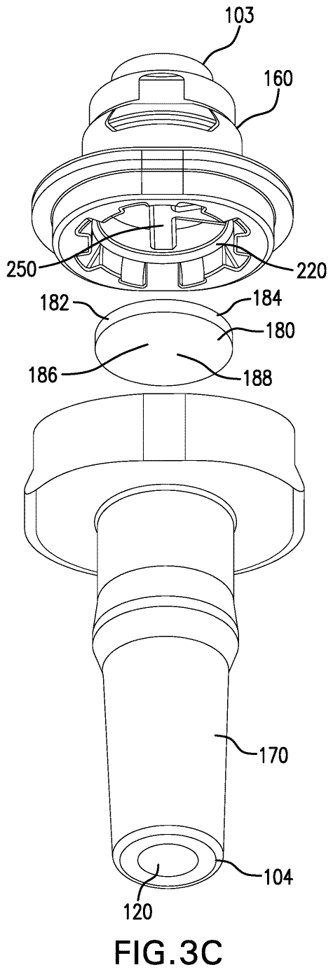

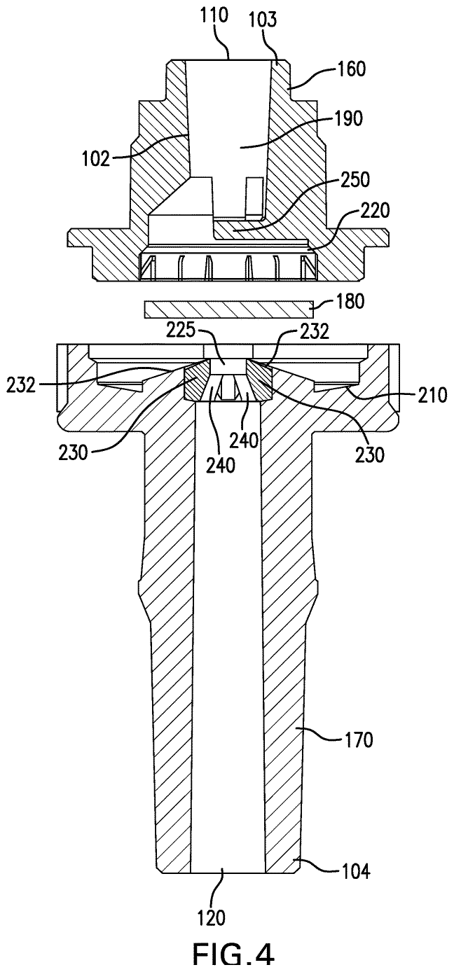

As noted above, to control fluid flow through the valve 10, the interior of the body/housing 100 may include a valve mechanism 180 within the internal fluid path 190. For example, the valve 10 may include a pressure activated valve 180 (PAV) that includes a flexible diaphragm 182 (e.g., a flat diaphragm; FIGS. 3A-3C and 4). The valve member 180 may have an outer section 184 that extends around the outer periphery of the valve member 180 and an inner section 186 located near/at the center of the valve member 180 and/or diaphragm 182. When in the closed mode (FIG. 2A), the valve mechanism 180 (e.g., the top surface of the outer portion 182) may seal against a seating/sealing surface 220 within the interior of the body/housing 100 (e.g., on the inner wall 102 of the body/housing 100). The valve mechanism/member 180 prevents fluid flow through the body/housing 100 (e.g., through the internal fluid path 190) until it is exposed to a large enough pressure or force (e.g., a forward pressure directed from the inlet 110 toward the outlet 120 or a backward pressure/force directed from the outlet 120 toward the inlet 110) to deform the diaphragm 182 (FIGS. 2B and 2C) and allow fluid to pass through the valve 10.

For example, in the presence of a forward pressure (e.g., a distally directed pressure from the inlet 110 toward the outlet 120), the valve member 180/diaphragm 182 may transition to/toward a first open mode (FIG. 2B). To that end, the valve member 180 may deform towards the distal end 104 of the valve 10 such that the outer section 184 of the diaphragm deforms away from the sealing surface 220 to allow fluid to flow around the valve member 180 from the inlet 110 toward the outlet 120). Conversely, in the presence of a sufficient backward/retrograde pressure or force (e.g., a proximally directed force from the outlet 120 toward the inlet 110), the valve member may transition to/toward a second open mode (FIG. 2C). As the valve member 180 transitions toward the second open mode, a portion of the valve member 180/diaphragm deforms toward the proximal end 103 of the valve 10, causing at least a portion of the outer section to slide past the sealing surface 220 and deform toward the inlet 110. This, in turn, allows fluid flow past the deformed portion of the outer section from the outlet 120 toward the inlet 110. The pressure required to transition the valve member 180 from the closed mode to the first and/or second open modes may depend on the application. However, in some embodiments, the forward pressure required to transition the valve member 180 toward the first open mode may be less than the pressure required to transition the valve member toward the second open mode. Additionally or alternatively, the pressure required to transition the valve 10 from the closed mode to the second open mode may be above the venous pressure of the patient.

In some embodiments and to help position the valve mechanism 180 within the fluid path 190 and provide a surface over which the valve member 180 may deform as the valve transitions toward the first open mode, the valve 10 may include a positioning structure 225 located on the distal side of the valve member 180 and within the fluid path 190. As the valve member 180 transitions toward the first open mode and/or in the presence of the distally directed pressure, the positioning structure 225 maintains the position of the inner section 186 of the valve member 180, but allows the outer section 184 to deform as discussed above.

Additionally, the valve 10 may have one or more protrusions 230 (FIGS. 3A and 3B) that extends proximally from the base portion 210 to form the positioning structure 225. The protrusion(s) 230 may contact the bottom surface 188 of the diaphragm 182 to position the valve 180 within the flow path 190 when in the first open mode. Although any number of configurations and lengths may be used for the protrusion(s) 230, in some embodiments, the protrusion(s) 230 may each have a similar length and width and may have angled radially outward faces 232. Alternatively, the protrusion(s) 230 may include a series of long protrusions 230A that extend and connect to form a portion of the positioning structure 225 and a series of shorter protrusions 230B that do not connect to form another portion of the positioning structure 225. To allow fluid flow between the protrusion(s) 230, the protrusion(s) 230 may be spaced from one another to create channels 240 between them. It should be noted that although FIG. 3B shows eight protrusion(s) 230 (four long protrusions 230A and four short protrusions 230B), other embodiments may utilize more or less than eight protrusions 230 and/or more or less than 4 short and long protrusions 230. In addition to the number of protrusion(s) 230, the width, length and spacing of the protrusion(s) 230 can vary based on the size of the internal fluid path 190 and the desired flow properties of the valve 10 (or system in which the valve 10 is incorporated).

As noted above, as the valve member 180 transitions from the closed mode to the second open mode, only a portion (e.g., a first portion) of the outer section 184 of the valve member 180 deforms. To that end and to prevent the rest of the outer section 184 (e.g., a second portion of the outer section 184) and at least a portion of the inner section 186 from deforming, the valve 10 may have a support structure 250 that is located within the fluid path proximal to the valve member 180 (e.g., as part/within the inlet housing 160). In the presence of the proximally directed force (e.g., a continuously applied force from the distal port 120 toward the proximal port 110 and/or a vacuum applied to the proximal end 103 of internal fluid path 190), the support structure 250 may support the second portion of the outer section 184 and the at least a portion of the inner section 186 of the valve member 180 to prevent the second portion (e.g., the supported portion) and the at least a portion of the inner section 186 from deforming under the pressure/force. Accordingly, only the first/unsupported portion of the outer section 184 will meaningfully deform as the valve member 180 transitions toward the second open mode. In this manner, the valve member 180 will deform asymmetrically as it transitions toward the second open mode (e.g., the first portion of the outer section 184 moves toward the proximal end 103 an amount greater than the second portion).

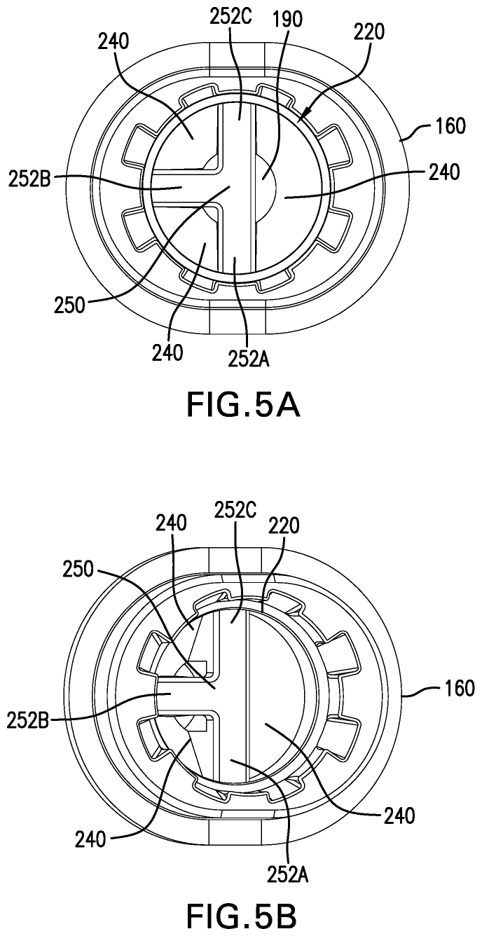

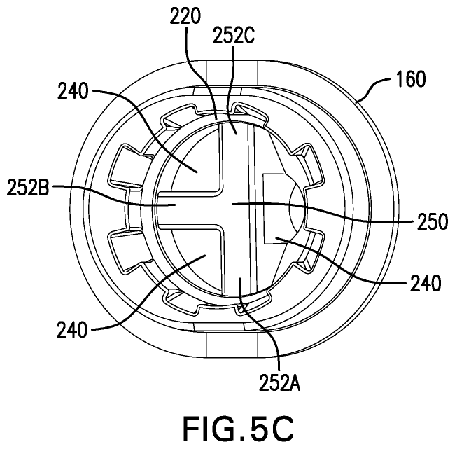

The support structure 250 can be any shape that sufficiently supports part of the outer section 184 and the at least a portion of the inner section 186, but allows enough of the outer section 184 (e.g., the first portion) to deform to allow a suitable/desired flow through the valve 10. As shown in FIGS. 3B and 5A-5C, the support structure 250 may be a T-shaped member that has support arms 252A-C that extend from the inner wall 102 (e.g., of the inlet housing 160) and meet in the center of the fluid path 190. These support arms 252A-C support the valve member 180 when in the second open mode. In other embodiments, the support structure 250 may be different shapes and/or configurations. For example, in other embodiments the support structure 250 may be V-shaped (e.g., two support arms that meet or nearly meet in the center to form a V), a cross shape (e.g., four support arms that meet or nearly meet in or near the center to form a cross), or bar shape (one arm that extends at least partially across the fluid path 190), to name but a few.

To allow fluid flow past the valve member 180 and the support structure 250, the support arms 252 may define at least one flow channel 253 through which fluid may pass. For example, as shown in FIGS. 3B and 5A-5C, the support arms 252 may be spaced from on another to create the flow channel(s) 253. Alternatively, if the support structure 250 only has a single support arm 252 (e.g., a single arm that extends entirely across the fluid path 190 or partially across the fluid path 190), the flow channels 253 may be located on either side of the single arm 252 (e.g., between the arm 252 and the inner wall 102). Accordingly, when the valve member 180 is in the first open mode, the fluid may flow through the flow channels 253, past the valve member 180, and out of the distal port/outlet 120.

It should be noted that, by supporting the valve member 180 on the proximal side such that only a portion of the outer section 184 deforms or inverts during retrograde flow, embodiments of the present invention are able to achieve a higher retrograde flow rate through a relatively large opening around the valve member 180 (as compared to the flow rate through a slit-style diaphragm of similar size and durometer) while maintaining the general location of the valve member 180 within the housing 100 and preserving the functionality of the valve 10. Of consequence, for fluid pathways used in IV applications, by supporting the valve member 180 in the manner described above, the opening formed around the valve member 180 during retrograde flow has a cross-sectional area that is an order of magnitude greater than the opening formed by a slit within a slit-style diaphragm of comparable size and durometer under the same flow conditions.

During operation, the user may connect a medical implement (e.g., a needleless syringe) to the inlet 110 of the valve 10 and begin to inject fluid. The forward pressure (e.g., a distally directed pressure) created by this fluid will cause the valve mechanism 180 to deform and cause the outer section 184 of the valve mechanism 180 to move away from the sealing surface 220 on the wall 102 of the body 100 and bend about the positioning structure 225. This, in turn, allows the fluid to flow from the first end of the body 100 (e.g., the inlet 110), past the seating/sealing surface 220, around the valve mechanism 180 into the channels 240, and towards the second end/outlet 120 of the body 100. Once in the first open mode, the user can transfer fluid to the patient. Conversely, if the user wishes to transfer fluid from the patient (e.g., for retrograde flow), the user may transition the valve member 180 to the second open mode by drawing back on the medical implement. This, in turn, creates a vacuum being pulled from the proximal end and a proximally directed force that causes the unsupported portion of the valve member 180/outer section 184 to deform past the sealing surface 220 to allow the fluid to flow from the second end/outlet 120 of the body 100 toward the first end/inlet 110 of the body 100.

To close the valve member 180 when it is in the first open mode, the operator may simply remove the distally directed pressure. This, in turn, will cause the valve member 180 to return to the closed mode. For example, the valve member 180 may be biased toward the closed mode such that the valve member 180 naturally returns to the closed mode or the venous pressure to which the valve member 180 is exposed may cause the valve member 180 to return toward the closed mode. Conversely, to transition the valve member 180 from the second open mode back to the closed mode (or to the first open mode), the operator may apply a distally directed pressure to the valve 10, which causes the valve member 180 to deform past the edge of the sealing surface 220. If the distally directed pressure is maintained, the valve member 180 will then transition to the first open mode. If the distally directed pressure is removed after the outer section 184 moves past the edge of the sealing surface 220, the valve member 180 will then transition to the closed mode in a manner similar to that described above. Furthermore, the amount of distally directed pressure required to move the outer section 184 of the valve member 180 back past the edge of the sealing surface 220 may be controlled by the amount of radial interference between the outer section 184 and sealing surface, the surface geometry and/or roughness of the inner wall 102, the thickness/durometer of the valve member 180, the angles between the inner wall 102 and edge of valve member 180, the lubricity of relevant surfaces, and/or other structural or frictional elements associated with the mechanics of the valve 10.

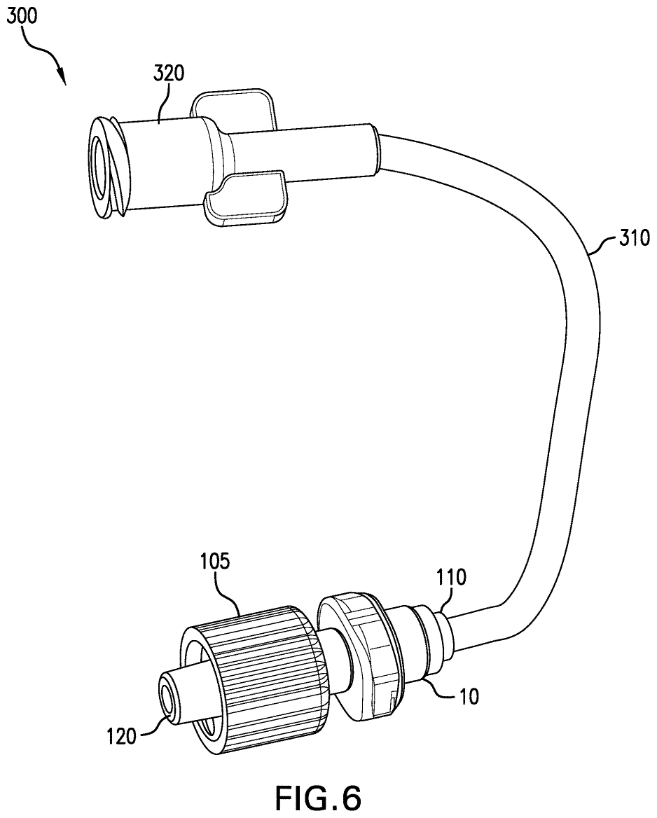

It should be noted that the valve 10 may be incorporated into any number of flow valving systems used within IV Therapy and Vascular Access devices. For example, as shown in FIG. 6, the valve 10 may be incorporated into an extension set 300. In such embodiments, a tube 310 of the extension set 300 may be inserted into and secured within the inlet 110 or alternatively, inserted into and secured within another component (e.g., a medical device and/or male luer connector) that, in turn, is connected to the inlet 110. For example, the tube 310 may be press-fit, ultrasonic welded, plastic welded, bonded, etc. within the inlet 110 or the component. During use, the medical implement (e.g., a needleless syringe) may be connected to a female luer 320 located on a longitudinal end of the tube 310 and the fluid may be injected into the valve 10 via the tube 310 and female luer connector 320.

Alternatively, the medical instrument may connect directly to the valve 10. To that end, the outside surface of the valve proximal port 110 may have inlet threads for connecting a medical instrument. Alternatively or in addition, the proximal end 103 may have a slip design for accepting instruments that do not have a threaded interconnect. In a similar manner, the distal end 104 of the valve 10 has a skirt 105 containing threads for connecting a threaded port of a catheter or a different medical instrument, to the valve distal port 120. If equipped, the proximal end inlet threads and the distal end threads would preferably comply with ANSI/ISO standards (e.g., they are able to receive/connect to medical instruments complying with ANSI/ISO standards). In addition to the threads described above, the internal geometry of the inlet housing 160 may have a taper and comply with ANSI/ISO standards.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A medical valve for use within devices in fluid communication with a patient's vasculature comprising:
a body defining the structure of the medical valve, the body having a first opening at a proximal end of the body, a second opening at a distal end of the body, and an internal fluid path extending from the first opening to the second opening;
a valve member located within the internal fluid path and having an outer section and an inner section, the valve member having a closed mode that prevents fluid flow through the medical valve, a first open mode that allows fluid flow from the first opening to the second opening, and a second open mode that allows fluid flow from the second opening to the first opening, the outer section deforming toward the distal end as the valve member transitions from the closed mode toward the first open mode in the presence of a distally directed force, at least a first portion of the outer section deforming toward the proximal end as the valve member transitions from the closed mode toward the second open mode in the presence of a proximally directed force, the valve member configured to transition from the second open mode to either the closed mode or the first open mode in the presence of a distally directed force;
a seal surface located on the body, the outer section of the valve member configured to seal against the seal surface when in the closed mode;
a positioning structure located within the fluid path distal to the valve member and configured to maintain the position of the inner section of the valve member in the presence of a continually applied distally directed force; and
a support structure located within the fluid path proximal to the valve member and configured to support at least a second portion of the valve member in the presence of a continually applied proximally directed force, wherein the outer section of the valve member deforming in an asymmetrical manner such that at least the first portion of the outer section of the valve member moves toward the proximal end of the body an amount greater than the second portion as the valve member transitions toward the second open mode.

2. A medical valve according to claim 1, wherein the body includes an inlet housing and an outlet housing, the first opening located in the inlet housing, the second opening located in the outlet housing.

3. A medical valve according to claim 2, wherein the seal surface and the support structure are located on the inlet housing.

4. A medical valve according to claim 2, wherein the positioning structure is located in the outlet housing and is configured to position the valve member between the inlet and outlet housing.

5. A medical valve according to claim 2, wherein the outer section of the valve member deforms toward the outlet housing as the valve member transitions toward the first open mode.

6. A medical valve according to claim 2, wherein the at least a portion of the outer section of the valve member deforms toward the inlet housing as the valve transitions toward the second open mode.

7. A medical valve according to claim 2, wherein the outlet housing includes at least one protrusion located within the outlet housing, the at least one protrusion positioning the valve member when in the first open mode and at least partly defining one or more flow channels through which fluid may flow.

8. A medical valve according to claim 2, wherein the support structure includes at least one support arm located within the inlet housing, the at least one support arm supporting the valve member when in the second open mode and at least partly defining one or more flow channels through which fluid may flow.

9. A medical valve according to claim 1, wherein the at least a first portion of the outer section is not supported by the support structure.

10. A medical valve according to claim 1, wherein the second portion of the valve member includes a second portion of the outer section and at least a portion of the inner section of the valve member.

11. A medical valve according to claim 1, wherein the valve member is a flexible diaphragm.

12. A medical valve according to claim 1, wherein the proximally directed force required to transition the valve member from the closed mode to the second open mode is greater than the distally directed pressure required to transition the valve member to the first open mode.

13. A medical valve according to claim 1, wherein the proximally directed force required to transition the valve member from the closed mode to the second open mode is greater than a patient's venous pressure.

14. A medical valve according to claim 1, wherein an amount of force required to move the outer section of the valve member past an edge of the sealing surface as the valve member transitions from the second open mode to either the closed mode or the first open mode is based on at least one selected from the group consisting of: an amount of radial interference between the outer section and the sealing surface, a surface geometry and/or roughness of an inner wall of the medical valve, a thickness and/or durometer of the valve member, one or more angles between the inner wall and an edge of valve member, and a lubricity of the surface of the valve member or inner wall.

15. A fluid management device in fluid communication with a patient's vasculature comprising:
a medical valve having:
a body defining the structure of the medical valve, the body having a first opening at a proximal end of the body, a second opening at a distal end of the body, and an internal fluid path extending from the first opening to the second opening,
a valve member located within the internal fluid path and having an outer section and an inner section, the valve member having a closed mode that prevents fluid flow through the medical valve, a first open mode that allows fluid flow from the first opening to the second opening, and a second open mode that allows fluid flow from the second opening to the first opening, the outer section deforming toward the distal end as the valve member transitions from the closed mode toward the first open mode in the presence of a distally directed force, at least a first portion of the outer section deforming toward the proximal end as the valve member transitions from the closed mode toward the second open mode in the presence of a proximally directed force, the valve member configured to transition from the second open mode to either the closed mode or the first open mode in the presence of a distally directed force,
a seal surface located on the body, the outer section of the valve member configured to seal against the seal surface when in the closed mode,
a positioning structure located within the fluid path distal to the valve member and configured to maintain the position of the inner section of the valve member in the presence of a continually applied distally directed force, and
a support structure located within the fluid path proximal to the valve member and configured to support at least a second portion of the valve member in the presence of a continually applied proximally directed force, wherein the outer section of the valve member deforming in an asymmetrical manner such that at least the first portion of the outer section of the valve member moves toward the proximal end of the body an amount greater than the second portion as the valve member transitions toward the second open mode; and
a tube having a first end and a second end, the first end fluidly connected to the first opening of the medical valve, the second end connected to a female luer connector.

16. A fluid management device according to claim 15, wherein the female luer connector is configured to connect to a medical implement configured to introduce fluid into or draw fluid from the patient through the medical valve.

17. A fluid management device according to claim 15, wherein the distal end of the medical valve is configured to connect to a catheter.

18. A method for controlling fluid flow through a medical valve comprising:
providing a medical valve having:
a body defining the structure of the medical valve, the body having a first opening at a proximal end of the body, a second opening at a distal end of the body, and an internal fluid path extending from the first opening to the second opening,
a valve member located within the internal fluid path and having an outer section and an inner section, the valve member having a closed mode that prevents fluid flow through the medical valve, a first open mode that allows fluid flow from the first opening to the second opening, and a second open mode that allows fluid flow from the second opening to the first opening, the outer section deforming toward the distal end as the valve member transitions from the closed mode toward the first open mode in the presence of a distally directed force, at least a first portion of the outer section deforming toward the proximal end as the valve member transitions from the closed mode toward the second open mode in the presence of a proximally directed force, the valve member configured to transition from the second open mode to either the closed mode or the first open mode in the presence of a distally directed force,
a seal surface located on the body, the outer section of the valve member configured to seal against the seal surface when in the closed mode,
a positioning structure located within the fluid path distal to the valve member and configured to maintain the position of the inner section of the valve member in the presence of a continually applied distally directed force, and
a support structure located within the fluid path proximal to the valve member and configured to support at least a second portion of the valve member in the presence of a continually applied proximally directed force, wherein the outer section of the valve member deforming in an asymmetrical manner such that at least the first portion of the outer section of the valve member moves toward the proximal end of the body an amount greater than the second portion as the valve member transitions toward the second open mode;
generating a pressure or force on the valve member to transition the valve member toward the first open mode or the second open mode; and
transferring fluid through the internal fluid path and around at least a portion of the valve member.

19. A method according to claim 18, wherein the pressure or force is a distally directed pressure and the valve member transitions toward the first open mode to allow fluid transfer to a patient and/or transition the valve member to the closed mode.

20. A method according to claim 18, wherein the pressure or force is a proximally directed force and the valve member transitions toward the second open mode to allow fluid transfer from a patient.
